(19) United States
(12) Patent Application Publication
(10) Pub. No.: US 2025/0187947 A1
(43) Pub. Date: Jun. 12, 2025

(54) FLUID STERILIZATION DEVICE

(71) Applicant: Stanley Electric Co., Ltd., Tokyo (JP)

(72) Inventors: Hideaki TANAKA, Tokyo (JP); Hiroyuki KATO, Tokyo (JP); Kazuhisa SHINNO, Tokyo (JP)

(21) Appl. No.: 18/843,241

(22) PCT Filed: Feb. 28, 2023
(86) PCT No.: PCT/JP2023/007283

§ 371 Date: Aug. 31, 2024

(30) Foreign Application Priority Data
Mar. 1, 2022 (JP) 2022-031288

(51) Int. Cl. C02F 1/32; A61L 2/10; H10H 29/24; H10H 29/856

(52) U.S. Cl. CPC C02F 1/325; A61L 2/10; H10H 29/24; H10H 29/856; A61L 2202/11; C02F 2201/3221; C02F 2201/3227; C02F 2201/3228; C02F 2303/04

(57) ABSTRACT

An aspect is to provide a fluid sterilization device that appropriately causes ultraviolet light emitted from a light source (light emitting element) to enter a flow path pipe to enhance sterilizing ability. The fluid sterilization device can include a cylindrical flow path pipe having a side peripheral wall, and a light source unit including a light emitting element disposed on the side peripheral wall and a substrate on which the light emitting element is mounted, wherein a notched region along a longitudinal direction of the flow path pipe is formed in the side peripheral wall of the flow path pipe, and the light source unit is housed in the notched region. The fluid sterilization device preferably further includes a heat sink that is disposed at a more radially outward position relative to the substrate of the light source unit and that is in contact with an outer surface of the substrate, and a housing that houses the heat sink. The flow path pipe preferably has a through hole at a position in the notched region where the light emitting element faces the notched region, and the fluid sterilization device preferably further includes: an ultraviolet light-transmitting window portion provided in the through hole; and an annular reflector that is housed in the notched region to surround the light emitting element.

This application is a U.S. National Stage Application under 35 U.S.C § 371 of International Patent Application No. PCT/JP2023/007283 filed Feb. 28, 2023, which claims the benefit of priority under 35 U.S.C. § 119 to Japanese Patent Application No. 2022-031288 filed Mar. 1, 2022, the disclosures of all of which are hereby incorporated by reference in their entireties.

### TECHNICAL FIELD

The presently disclosed subject matter relates to a fluid sterilization device.

### BACKGROUND ART

Various products have been provided for sterilizing fluids (e.g., liquid for beverages or raw water for food, or various types of cooling water and washing water for factories) by irradiation with ultraviolet light. Among inventions related to these products, a fluid sterilization device provided in a fluid flow path is disclosed in, for example, Patent Literature 1 below.

The fluid sterilization device disclosed in Patent Literature 1 includes a flow path pipe, a light source that irradiates the water passing through the flow path pipe with ultraviolet light, and a housing that houses the flow path pipe and the light source. Further, at least a part of the wall surface of the flow path pipe is provided with a window portion that transmits ultraviolet light, and the light source is provided at a position corresponding to the window portion.

### CITATION LIST

### Patent Literature

Patent Literature 1: Japanese Patent Application Laid-Open No. 2014-233646

### SUMMARY

### Technical Problem

However, in the fluid sterilization device disclosed in Patent Literature 1, the light source housed in the housing is first fixed to the housing outside the flow path pipe, and the housing is additionally fixed to the flow path pipe. When the device with such a structure is assembled, the number of assembly steps is large.

In view of the above-mentioned problem, one of the aspects of the presently disclosed subject matter is to provide a fluid sterilization device in which a light source unit (light emitting element) can be easily attached to a flow path pipe.

### Solution to Problem

In order to solve the above-mention problem, a fluid sterilization device according to the presently disclosed subject matter includes:
a cylindrical flow path pipe having a side peripheral wall; and
a light source unit including a light emitting element disposed on the side peripheral wall and a substrate on which the light emitting element is mounted, wherein
a notched region along a longitudinal direction of the flow path pipe is formed in the side peripheral wall of the flow path pipe, and
the light source unit is housed in the notched region.

According to this aspect of the presently disclosed subject matter, since the notched region for accommodating the light source unit is formed in the side peripheral wall of the flow path pipe, the light source unit (light emitting element) can be easily attached to the flow path pipe. Further, since the light emitting element is housed in a recessed space corresponding to the notched region, it is possible to cause the ultraviolet light emitted from the light emitting element to enter, without the light leaking out, the inside of the flow path pipe.

Further, the fluid sterilization device according to the presently disclosed subject matter preferably further includes:
a heat sink that is disposed at a more radially outward position relative to the substrate of the light source unit and that is in contact with an outer surface of the substrate, and
a housing that houses the heat sink.

According to this aspect of the presently disclosed subject matter, the heat sink can be brought into contact with the outer surface of the substrate of the light source unit, and the heat sink can be housed in the housing. Therefore, according to this aspect of the presently disclosed subject matter, it is possible to reduce the size of the fluid sterilization device and to appropriately dissipate the heat that is generated in the light source unit.

Further, in the fluid sterilization device according to the presently disclosed subject matter, the flow path pipe may preferably have a through hole at a position in the notched region where the light emitting element faces the notched region, and
the fluid sterilization device may preferably further include:
an ultraviolet light-transmitting window portion provided in the through hole; and
an annular reflector that is housed in the notched region to surround the light emitting element.

According to this aspect of the presently disclosed subject matter, the through hole is formed at a position in the notched region of the flow path pipe where the light emitting element faces the notched region, and the light source unit, the reflector, and the ultraviolet light-transmitting window portion can be housed in the notched region. As a result, the light emitting element, the reflector, and the ultraviolet light-transmitting window portion are located on an inner side of the flow path pipe. With this configuration, it is possible to further reduce the size of the fluid sterilization device and further increase the entrance efficiency in terms of how much ultraviolet light that is emitted from the light emitting element enters the flow path pipe.

Further, in the fluid sterilization device according to the presently disclosed subject matter, it is preferable that:
the light emitting element includes a plurality of light emitting elements, the reflector includes a plurality of reflectors, and the ultraviolet light-transmitting window portion includes a plurality of ultraviolet light-transmitting windows;
the flow path pipe has a plurality of through holes;
a first through hole among the through holes is provided at a first position of the side peripheral wall of the flow path pipe;
in the side peripheral wall, a second through hole among the through holes is provided at a second position a predetermined distance away from the first position in a circumferential direction;
at least one light emitting element among the plurality of light emitting elements, at least one reflector among the plurality of reflectors, and at least one ultraviolet light-transmitting window portion among the plurality of ultraviolet light-transmitting window portions face the first through hole; and
other light emitting elements among the plurality of light emitting elements, other reflectors among the plurality of reflectors, and other ultraviolet light-transmitting window portions among the plurality of ultraviolet light-transmitting window portions face the second through hole.

According to this aspect of the presently disclosed subject matter, it is possible to irradiate flowing water with ultraviolet light from a plurality of locations in the circumferential direction of the flow path pipe. As a result, the sterilizing ability can be further enhanced.

### Advantageous Effects of the Presently Disclosed Subject Matter

According to the presently disclosed subject matter, it is possible to provide a fluid sterilization device in which a light source unit (light emitting element) can be easily attached to a flow path pipe.

### BRIEF DESCRIPTION OF DRAWINGS

FIG. 1 is a vertical cross-sectional view of a fluid sterilization device according to an embodiment of the presently disclosed subject matter.

FIG. 2 is an exploded perspective view of the fluid sterilization device according to the present embodiment.

FIG. 3 is an exploded perspective view of the fluid sterilization device according to the present embodiment.

FIG. 4 (a) is a perspective view of a fluid sterilization device according to a modified example, and FIG. 4 (b) is a transverse cross-sectional view of the fluid sterilization device according to the modified example.

FIG. 5 is a vertical cross-sectional view of a fluid sterilization device according to another modified example.

### DESCRIPTION OF EMBODIMENTS

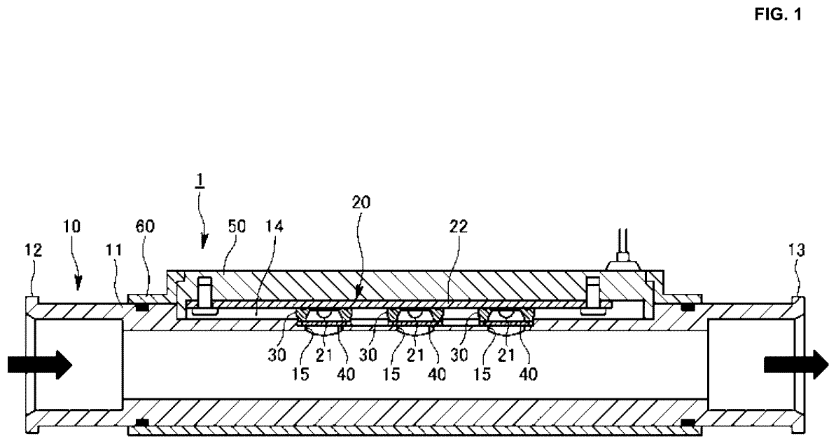

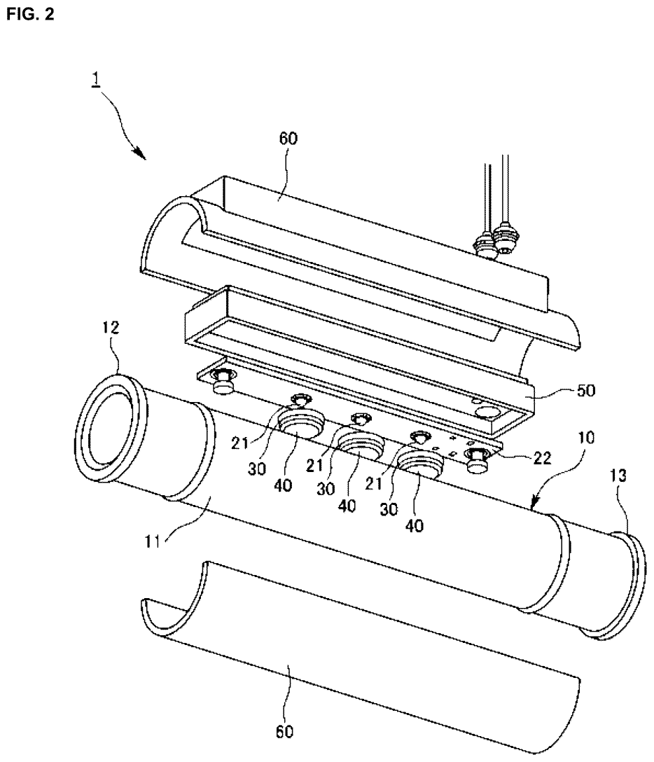

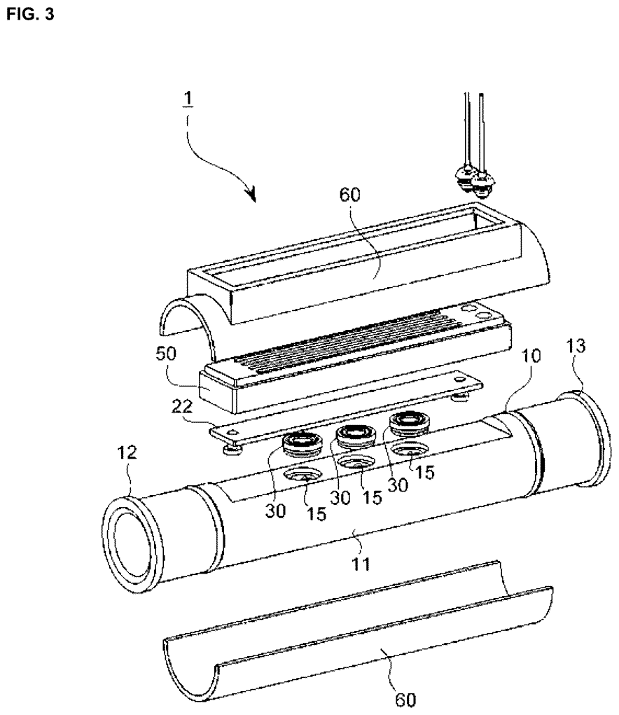

Hereinafter, a fluid sterilization device according to an embodiment of the presently disclosed subject matter will be described in detail with reference to the drawings. First, a fluid sterilization device 1 according to the present embodiment will be described with reference to FIGS. 1 to 3. Here, FIG. 1 is a vertical cross-sectional view of the fluid sterilization device 1. FIG. 2 is an exploded perspective view of the fluid sterilization device 1 as viewed from an obliquely lower side. Further, FIG. 3 is an exploded perspective view of the fluid sterilization device 1 as viewed from an obliquely upper side.

As illustrated in FIGS. 1 to 3, the fluid sterilization device 1 according to the present embodiment includes a flow path pipe 10 through which a fluid flows, a light source unit 20, reflectors 30, ultraviolet light-transmitting window portions (for example, quartz glass) 40, a heat sink 50, a housing 60, and the like.

The flow path pipe 10 of the present embodiment is a tubular member having a side peripheral wall 11. Further, the flow path pipe 10 has a straight pipe shape (I-shape), and further has open end portions (an inlet end 12 and an outlet end 13) at the two ends opposite to each other in a longitudinal direction. Thus, the fluid to be sterilized (e.g., water) flows into the flow path pipe 10 from the inlet end 12, flows within the confines of the side peripheral wall 11, and flows out from the outlet end 13. However, the configuration of the flow path pipe 10 is not limited to this. Examples of other configurations of the flow path pipe 10 include an L-shaped configuration.

Further, as shown in FIGS. 1 to 3, the flow path pipe 10 of the present embodiment includes a notched region 14 that is along the longitudinal direction in the side peripheral wall 11 and that is recessed radially inward relative to the flow path pipe 10 (side peripheral wall 11), and through holes 15 that penetrate through to the notched region 14 of the side peripheral wall 11. As will be described later, light emitting elements 21 of the light source unit 20 face the through holes 15 provided in the notched region 14.

The flow path pipe 10 of the present embodiment has three through holes 15, but the number of the through holes 15 is not limited to this. The flow path pipe 10 of the present embodiment is made of polytetrafluoroethylene (PTFE). However, the material of the flow path pipe 10 is not limited to this. Other materials of the flow path pipe 10 include a perfluoroethylene propene copolymer (FEP), perfluoroalkoxyalkane (PFA), and the like.

Next, as illustrated in FIGS. 1 to 3, the light source unit 20 include the light emitting elements 21 that emit ultraviolet light (for example, light having a peak wavelength in a range of 100 nm to 400 nm), a substrate 22 on which the light emitting elements 21 are mounted, and the like.

The light source unit 20 of the present embodiment is disposed so as to face the notched region 14 of the flow path pipe 10 and to fit inside the notched region 14. Further, as described above, the light emitting elements 21 face the through holes 15 provided in the notched region 14. As a result, the ultraviolet light from the light emitting elements 21 enters, without the light leaking out, the inside of the flow path pipe 10 (the inside of the side peripheral wall 11), and is emitted toward the fluid flowing through the flow path pipe 10.

Since the light source unit 20 and the heat sink 50 are placed so as to be fitted into the notched region 14, they are less likely to experience positional deviation, even during the assembly operation before being fixed by the housing 60. Therefore, it is possible to easily perform an assembly process in which the light source unit 20 and the heat sink 50 are placed in the notched region 14 and sandwiched by the housing 60. Further, while the flow path pipe 10 is cylindrical, the placement surface for the light source unit 20 in the notched region 14 is formed in a planar shape. Therefore, it is easy to attach the substrate 22 and the heat sink 50 to the notched region 14.

The type of light emitting elements 21 is not particularly limited, and examples thereof include an LED (Light emitting diode) and a semiconductor light emitting element such as a laser diode. In addition, three light emitting elements 21 of the present embodiment are provided in line along the longitudinal direction, but the presently disclosed subject matter is not limited thereto. Further, in the present embodiment, one light emitting element 21 faces one through hole 15, but a plurality of light emitting elements 21 may face one through hole 15.

As described above, since the light emitting elements 21 are disposed so as to face the through holes 15 of the flow path pipe 10, the ultraviolet light emitted from the light emitting elements 21 can be caused to enter, without the light leaking out, the inside of the flow path pipe 10. As a result, the entrance efficiency, in terms of how much ultraviolet light that is emitted from the light emitting elements 21 enters the flow path pipe 10, can be increased, and the sterilizing ability can thus be improved.

In addition, since the light emitting elements 21 face the through holes 15 formed in the notch region 14, which is recessed radially inward relative to the flow path pipe 10 (the side peripheral wall 11), the light emitting elements 21 can be disposed on the inner side of the flow path pipe 10. Accordingly, the substrate 22 on which the light emitting elements 21 are mounted is also disposed on the inner side of the flow path pipe 10. As a result, the size of the fluid sterilization device 1 can be reduced, and the entrance efficiency in terms of how much ultraviolet light that is emitted from the light emitting elements 21 enters (the efficiency of entrance into the flow path pipe 10) can be further increased.

Next, as shown in FIG. 1, the reflector 30 is an annular member surrounding the light emitting element 21. Further, the reflector 30 of the present embodiment is located between the substrate 22 and the side peripheral wall 11 of the flow path pipe 10, so that the reflector 30 is housed in the notched region 14 of the flow path pipe 10. According to the present embodiment, the provision of the reflector 30 can distribute the ultraviolet light so that the ultraviolet light from the light emitting element 21 is emitted in a desired range. As a result, the illuminance of the ultraviolet light in the light distribution range can be increased, and the sterilizing ability can further be improved.

Next, as shown in FIG. 1, the ultraviolet light-transmitting window portion 40 is sandwiched between the peripheral edge of the through hole 15 and the front end of the reflector 30 in the notched region 14 of the flow path pipe 10. By sandwiching the ultraviolet light-transmitting window portion 40 between the peripheral edge of the through hole 15 and the front end of the reflector 30 which are accommodated in the notched region 14, the flowing water sterilization device 1 can be further reduced in size.

Next, as shown in FIGS. 1 to 3, the heat sink 50 is disposed at a more radially outward position relative to the substrate 22 of the light source unit 20. More specifically, the heat sink 50 is in contact with an outer surface (a surface facing radially outward) of the substrate 22. Further, the bottom side of the heat sink 50 fits into the notched region 14 of the flow path pipe 10.

By disposing the heat sink 50 in this manner, the heat generated in the light source unit 20 (the light emitting elements 21 and the substrate 22) can be appropriately dissipated, and the flowing water sterilization device 1 can be further reduced in size.

Next, the housing 60 is a cylindrical member circumferentially provided on the flow path pipe 10 (side peripheral wall 11). As shown in FIGS. 1 to 3, the housing 60 houses the heat sink 50 therein. The housing 60 of the present embodiment is made of a resin such as polypropylene (PP), but is not limited thereto.

As described above, by housing the heat sink 50 in the cylindrical housing 60, a situation in which the heat sink 50 is detached from the flow path pipe 10 can be prevented. Further, it is possible to further reduce the size of the flowing water sterilization device 1.

An embodiment of the presently disclosed subject matter has been described in detail above. However, the foregoing description is for the purpose of facilitating understanding of the presently disclosed subject matter, and is not intended to limit the presently disclosed subject matter. The present invention may include changes and improvements without departing from the spirit thereof. The present invention also includes equivalents thereof.

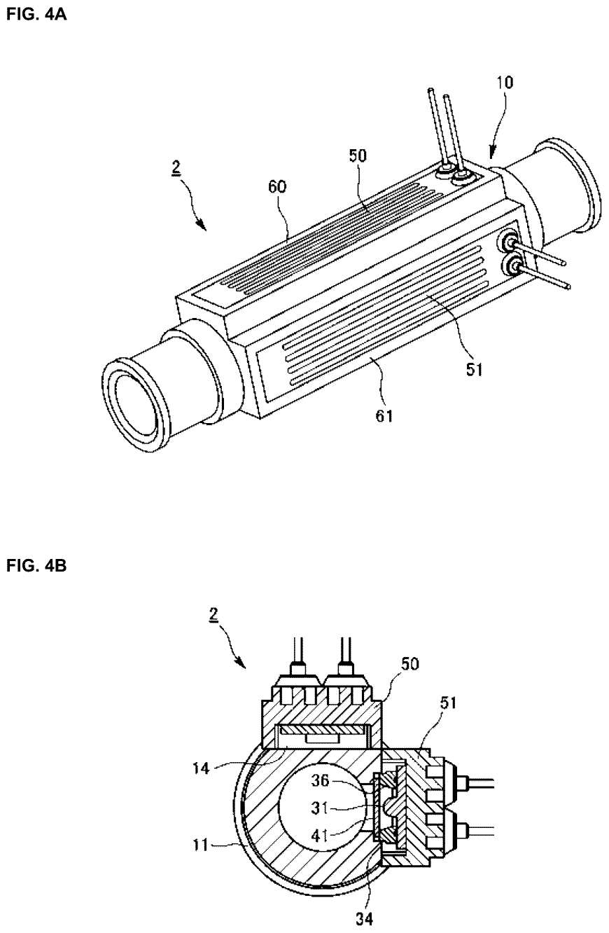

In the fluid sterilization device 1 according to the above-described embodiment, one notch region 14 of the flow path pipe 10 is formed, but the device may be a fluid sterilization device 2 having a structure as shown in FIG. 4 (hereinafter, the fluid sterilization device 2 shown in FIG. 4 may be referred to as a “modified example” in some cases).

Here, FIG. 4 (a) is a perspective view of the fluid sterilization device 2 according to the modified example, and FIG. 4 (b) is a transverse cross-sectional view of the fluid sterilization device 2 according to the modified example. As shown in FIG. 4 (b), in the fluid sterilization device 2 according to the modified example, notched regions 14 and 34 may be formed at a plurality of positions of the flow path pipe 10 (the notched region 14 and the notched region 34 are located a predetermined distance away from each other in the circumferential direction of the side peripheral wall 11).

Hereinafter, the notched region 14 is referred to as a first notched region 14, and the notched region 34 is referred to as a second notched region 34. Further, in the side peripheral wall 11 of the flow path pipe 10, a position where the first notched region 14 is formed is referred to as a first position, and a position where the second notched region 34 is formed is referred to as a second position.

The positional relationship between the first notched region 14 (first position) and the second notched region 34 (second position) is not particularly limited, but in the case of the modified example shown in FIG. 4, the second notched region 34 is provided at a position rotated 90° clockwise from the first notched region 14 with the center of the side peripheral wall 11 as an origin.

In addition, the through hole 15 penetrates through to the first notched region 14. Further, another through hole 35 penetrates through to the second notched region 34.

Hereinafter, the through hole 15 that penetrates through to the first notched region 14 is referred to as a first through hole 15, and the through hole 35 that penetrates through to the second notched region 34 is referred to as a second through hole 35.

Further, the light emitting element 21 (light source unit), the reflector 30, and the ultraviolet light-transmitting window portion 40 face the first through hole 15. Further, a light emitting element 31 (light source unit) different from the light emitting element 21, a reflector 36 different from the reflector 30, and an ultraviolet light-transmitting window portion 41 different from the ultraviolet light-transmitting window portion 40 face the second through hole 35.

The number of the light emitting elements 21 and the like (the sets consisting of the light emitting element 21, the reflector 30, and the ultraviolet light-transmitting window portions 40) provided in the first notched region 14 (the first position) is not particularly limited. The number of the light emitting elements 31 and the like (the sets consisting of the light emitting element 31, the reflector 36, and the ultraviolet light-transmitting window portion 41) provided in the second notched region 34 is not particularly limited.

Further, as shown in FIG. 4 (a), a heat sink 51 different from the heat sink 50 is provided. The heat sink 50 is in thermal contact with the light emitting element 21 (the substrate 22 on which the light emitting element 21 is mounted), and the heat sink 51 is in thermal contact with the light emitting element 31 (the substrate on which the light emitting element 31 is mounted). Further, in addition to the housing 60 that houses the heat sink 50, another housing 61 that houses the heat sink 51 is provided.

According to the above-described modified example, it is possible to irradiate the flowing water with ultraviolet light from a plurality of locations in the circumferential direction of the flow path pipe 10. As a result, the illuminance of the ultraviolet light emitted to the fluid can be increased, and the sterilizing ability can thus be further increased.

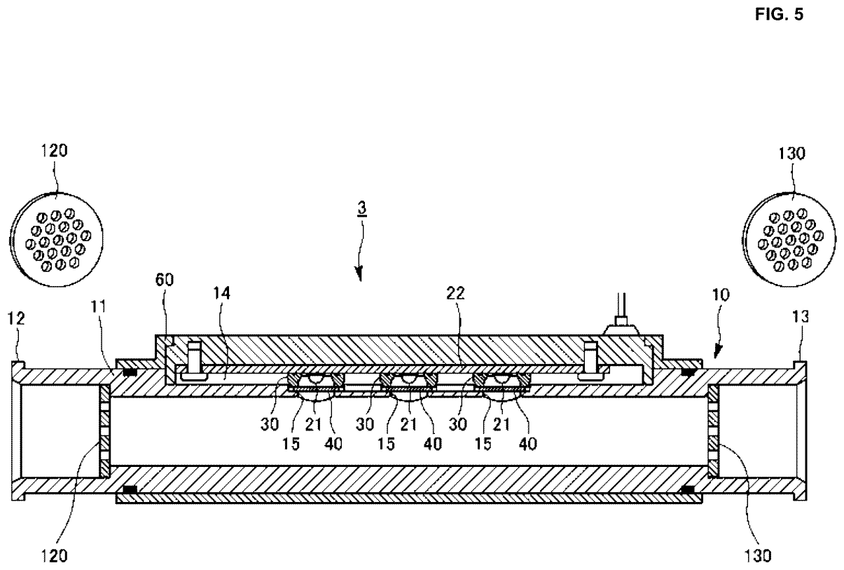

Further, as in another modified example (fluid sterilization device 3) shown in FIG. 5, water restriction plates 120 and 130 having a plurality of small holes may be disposed at each of the inlet end 12 and the outlet end 13 sides of the flow path pipe 10.

### INDUSTRIAL APPLICABILITY

The fluid sterilization device according to the presently disclosed subject matter is used for, for example, an ultraviolet light sterilization device, a water cleaner, a hot water supply device, a water supply pipe, a cooling water circulation device, a water server, a drink server, or the like. However, the use thereof is not limited to these.

### REFERENCE SIGNS LIST

1 . . . fluid sterilization device
10 . . . flow path pipe
11 . . . side peripheral wall
12 . . . inlet end
13 . . . outlet end
14, 34 . . . notched region
20 . . . light source unit
21, 31 . . . light emitting element
22 . . . substrate
30, 36 . . . reflector
40, 41 . . . ultraviolet light-transmitting window portion
50, 51 . . . heat sink
60, 61 . . . housing

1. A fluid sterilization device comprising:
a cylindrical flow path pipe having a side peripheral wall; and
a light source unit including a light emitting element disposed on the side peripheral wall and a substrate on which the light emitting element is mounted, wherein
a notched region along a longitudinal direction of the flow path pipe is formed in the side peripheral wall of the flow path pipe, and
the light source unit is housed in the notched region.

2. The fluid sterilization device according to claim 1, further comprising:
a heat sink that is disposed at a radially outward position relative to the substrate of the light source unit and that is in contact with an outer surface of the substrate, and
a housing that houses the heat sink.

3. The fluid sterilization device according to claim 1- or 2,
wherein:
the flow path pipe has a through hole at a position in the notched region where the light emitting element faces the notched region; and
the fluid sterilization device further comprises:
an ultraviolet light-transmitting window portion provided in the through hole; and
an annular reflector that is housed in the notched region to surround the light emitting element.

4. The fluid sterilization device according to claim 3, wherein:
the light emitting element includes a plurality of light emitting elements, the reflector includes a plurality of reflectors, and the ultraviolet light-transmitting window portion includes a plurality of ultraviolet light-transmitting windows;
the flow path pipe has a plurality of through holes;
a first through hole among the through holes is provided at a first position of the side peripheral wall of the flow path pipe;
in the side peripheral wall, a second through hole among the through holes is provided at a second position a predetermined distance away from the first position in a circumferential direction;
at least one light emitting element among the plurality of light emitting elements, at least one reflector among the plurality of reflectors, and at least one ultraviolet light-transmitting window portion among the plurality of ultraviolet light-transmitting window portions face the first through hole; and
other light emitting elements among the plurality of light emitting elements, other reflectors among the plurality of reflectors, and other ultraviolet light-transmitting window portions among the plurality of ultraviolet light-transmitting window portions face the second through hole.

5. The fluid sterilization device according to claim 2, wherein:
the flow path pipe has a through hole at a position in the notched region where the light emitting element faces the notched region; and
the fluid sterilization device further comprises:
an ultraviolet light-transmitting window portion provided in the through hole; and
an annular reflector that is housed in the notched region to surround the light emitting element.

6. The fluid sterilization device according to claim 5, wherein:
the light emitting element includes a plurality of light emitting elements, the reflector includes a plurality of reflectors, and the ultraviolet light-transmitting window portion includes a plurality of ultraviolet light-transmitting windows;
the flow path pipe has a plurality of through holes;
a first through hole among the through holes is provided at a first position of the side peripheral wall of the flow path pipe;
in the side peripheral wall, a second through hole among the through holes is provided at a second position a predetermined distance away from the first position in a circumferential direction;
at least one light emitting element among the plurality of light emitting elements, at least one reflector among the plurality of reflectors, and at least one ultraviolet light-transmitting window portion among the plurality of ultraviolet light-transmitting window portions face the first through hole; and
other light emitting elements among the plurality of light emitting elements, other reflectors among the plurality of reflectors, and other ultraviolet light-transmitting window portions among the plurality of ultraviolet light-transmitting window portions face the second through hole.
